(19) United States
(12) Patent Application Publication
(10) Pub. No.: US 2006/0190070 A1
(43) Pub. Date: Aug. 24, 2006

(54) Rail stent and methods of use

(76) Inventors: Martin S. Dieck, Cupertino, CA (US); Brian B. Martin, Boulder Creek, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/360,744

(22) Filed: Feb. 22, 2006

Related U.S. Application Data
(60) Provisional application No. 60/655,525, filed on Feb. 23, 2005.

(51) Int. Cl. A61F 2/06

(52) U.S. Cl. 623/1.12

(57) ABSTRACT

Devices, systems and methods are provided for stenting body lumens. In particular, stents are provided which are advanceable directly over a guidewire and expandable within a target location of a body lumen by retraction of the guidewire and/or by releasing constraining element(s) disposed around at least a portion of the stent. Typically the constraining element(s) have the form of one or more bands or layers of material which hold the stent in an unexpanded configuration. These stent designs allow delivery to a body lumen without the need for a number of additional devices which are typically used in the delivery of conventional stents, thereby reducing the profile of the stent during delivery, increasing the flexibility of the stent during delivery to allow passage through more tortuous pathways, and allowing the delivery of branched or otherwise connected stents to body lumens, such as branched lumens.

### CROSS-REFERENCES TO RELATED APPLICATIONS

This application claims the benefit and priority of U.S. Provisional Patent Application No. 60/655,525 (Attorney Docket TSU-001), filed Feb. 23, 2005, the full disclosure of which is hereby incorporated by reference for all purposes.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

A stent comprises a small metal coil, slotted tube, mesh or scaffold structure that is placed in a body lumen, such as the vasculature, to support the lumen wall. Such support may be desired in a variety of applications. For example, stents may be used following percutaneous transluminal coronary angioplasty (PTCA) procedures. In PTCA procedures, a catheter having a small balloon disposed near its distal end is advanced through the aorta and into a coronary artery that is at least partially occluded by arterial plaque. The balloon is then inflated, compressing the plaque against the arterial walls and restoring blood flow to the heart. A stent may be positioned to hold the artery open and prevent restenosis of the artery.

Stents may also be used to treat aneurysms. An aneurysm is a focal abnormal dilation of a blood vessel. The complications which arise from aneurysms include rupture, embolization and symptoms related to pressure on surrounding structures. Aneurysms are commonly found in the abdominal aorta, being that part of the aorta which extends from the diaphragm to the point at which the aorta bifurcates into the common iliac arteries. These abdominal aortic aneurysms typically occur between the point at which the renal arteries branch from the aorta and the bifurcation of the aorta. Aneurysms are also commonly found in the cerebral vasculature. Cerebral aneurysms are enlargements of the cerebral vasculature which protrude like a balloon from the wall of a cerebral artery. The cerebral aneurysm typically has a neck which leads to the parental vessel and a body or “dome” which can vary in diameter from 1-30 mm.

When left untreated, aneurysms may eventually rupture, often with ensuing fatal hemorrhaging in a very short time. Therefore, a variety of treatments have been developed, many of which involve positioning a stent within the blood vessel extending along the length of the aneurysm. For example, aneurysms may be treated by positioning a graft, comprised of a Dacron® polyester or a Teflon® polytetrafluoroethylene material, along the aneurysm site to reconstruct the dilated vessel. Often a stent is used to hold such a graft in place. Alternatively or in addition, aneurysms may be treated by filling the aneurysm with a packing material, such as platinum coils, and positioning a stent across the aneurysm to hold the packing materials therein. The packing materials are desired to promote thrombus within the aneurysm and eventually eliminate the threat of ruptures and promote resorption of the aneurysm. Other types of treatments may also be used.

Conventional stents have taken two forms, balloon expandable stents and self-expanding stents. Both are typically made of metallic materials and may include a biocompatible coating. Such stents are permanently implanted into the human body by deploying them on or through a catheter.

For balloon expandable stents, the stent is crimped around a collapsed balloon on a catheter in an unexpanded state. The unexpanded stent is then percutaneously inserted into the blood vessel using the catheter (such as an over-the-wire or Monorail type) and is guided to the site where it is to be permanently implanted. Upon reaching the site of implantation, the balloon portion of the catheter is expanded, and concomitantly the stent also is expanded as a result of the mechanical force applied by the expanding balloon until the stent is sized appropriately for the implantation site. Thereafter, the expanded balloon is deflated, and the catheter is removed from the body, leaving the stent held permanently in position. Balloon expandable stents are typically made from various metals and metal alloys, and take the form of slotted tube design, helical designs, wire design etc.

For self-expandable stents, the unexpanded stent is also percutaneously inserted into the body with the use of a catheter or sheath where it is guided to the site of implantation. However, the self-expanding stent, which may be a woven, slotted tube design or wound like a spring, is compressed within a catheter or sheath. The stent then is released from the interior of the sheath, where it expands to a fixed, predetermined diameter and is held in position as a result of that expansion.

Both balloon expanding and self-expanding conventional stents utilize a catheter or sheath for delivery. Typically, such delivery is achieved by a puncture of the femoral artery at the groin and placement of a femoral sheath by standard techniques, such as by the Seldinger Technique. A guide wire having a flexible tip is then advanced into the vessel. Under manual compression the needle is withdrawn and the delivery catheter is advanced over the guide wire into the artery and positioned at the desired location. In the case of balloon expanding stents, the balloon in then inflated to expand and deliver the stent. In the case of self-expanding stents, a sheath is typically withdrawn to expel the stent within the blood vessel wherein the stent self-expands.

A number of drawbacks are associated with such delivery. To begin, the above described delivery methods involve the use of a sheath or catheter, and a balloon in the case of a balloon expanding stent, in addition to the guidewire and stent itself. Such elements in their conventional form add considerable bulk and dimension to the delivery device creating a substantial outer diameter. This limits the size, type and location of vessels within which a stent may be placed. This is particularly problematic within the cerebral vasculature where blood vessels form tortuous pathways and have small diameters. In addition, the above described delivery methods restrict the use of bifurcated, branched or connected stents. Many aneurysms are located near bifurcations or branches in the vasculature, thus requiring placement of a stent having a similar configuration. However, in the case of self-expanding stents, such branched or connected stents are not configured for collapsing within a cylindrical sheath and expelling therefrom as described above. In addition, considerable force is required to deploy the stent from the catheter or sheath, which may be difficult to transmit through long tortuous pathways and may inhibit proper placement of the stent.

Therefore, a significantly low-profile delivery system is desired for the delivery of stents to the vasculature or to any suitable body lumen. In addition, a reconfigurable delivery system is desired to successfully deliver branched or otherwise connected stents to a body lumen, such as a branched or bifurcated lumen. Such delivery systems should be easy to use, cost effective and provide proper placement of a stent in a variety of locations, including small vessels previously prohibited by the size restrictions of conventional delivery catheters. At least some of these objectives will be met by the aspects of the present invention.

### BRIEF SUMMARY OF THE INVENTION

Devices, systems and methods are provided for stenting body lumens. In particular, for stenting body lumens having tortutous pathways, small diameters, bifurcated or branched configurations, and/or various types of aneurysms. Such body lumens include but are not limited to arteries, veins, biliary ducts, urethras, fallopian tubes, bronchial tubes, the trachea, the esophagus and the prostate. A specific use of the present invention is for the treatment of cerebral aneurysms although the various aspects of the invention described below may also be useful in treating any lumen which may benefit from the positioning of a stent therein, including abnormalities such as arteriovenous malformations (AVM), cavernous carotid fistulas, and non-reversible sterilization via fallopian tube occlusion.

The present invention provides stents which are advanceable directly over an elongate structure, such as a guidewire, and expandable within a target location of a body lumen by retraction of the structure and/or by releasing constraining element(s) disposed around at least a portion of the stent. It may be appreciated that guidewire shall be used interchangeably with elongate structure throughout. Typically the constraining element(s) have the form of one or more bands or layers of material which hold the stent in an unexpanded configuration. These stent designs allow delivery to a body lumen without the need for a number of additional devices which are typically used in the delivery of conventional stents, thereby reducing the profile of the stent during delivery. In particular, the need for a conventional delivery catheter, or any delivery catheter, is eliminated. Since the stents are advanceable directly over a guidewire, there is no need to mount the stent on or within a conventional delivery catheter which is then advanced over the guidewire. Such delivery catheters include external sheaths which are typically used to deliver self-expanding stents. Elimination of the conventional delivery catheter not only increases the flexibility of the stent during delivery to allow passage through more tortuous pathways, but also allows the delivery of branched or otherwise connected stents to body lumens, such as branched lumens. Examples of branched stents and body lumens provided herein focus on lumens that are bifurcated or have two branches, however it may be appreciated that stents and body lumen may have any number of branches including three, four, five, six, seven, eight or more. It may also be appreciated that stents of the present invention include a stent, graft, stent-graft, vena cava filter or other implantable medical device hereinafter collectively referred to generally as stents.

In a first aspect of the present invention, a stent is provided for positioning within a body lumen wherein the stent includes a radially expandable body having a first end, a second end and a longitudinal axis extending between the first and second ends and at least one loop having an opening extending from the first end. The expandable body is transitionable between an unexpanded state and an expanded state, and alignment of the opening of the at least one loop with the longitudinal axis transitions at least the first end toward the unexpanded state. Typically, the at least one loop is configured for passage of at least one guidewire therethrough. In such instances, the expandable body is advanceable directly over the at least one guidewire.

In some embodiments, the at least one loop comprises a plurality of loops extending around a circumference of the first end. Expandable bodies may further comprise at least one loop having an opening extending from the second end, wherein alignment of the opening of the at least one loop extending from the second end with the longitudinal axis transitions the second end toward the unexpanded state. Expandable bodies may further comprise a third end and another longitudinal axis extending between the first and third ends. In such embodiments, at least one loop having an opening extends from the third end, wherein alignment of the opening of the at least one loop extending from the third end with the another longitudinal axis transitions the third end toward the unexpanded state. Typically, the at least one loop of the third end is configured for passage of at least one guidewire therethrough. Thus, the expandable body may be simultaneously advanced directly over a first guidewire passed through the first and second ends and a second guidewire passed through the first and third ends.

In some embodiments, the expandable body comprises a frame formed from a plurality of wires. In such embodiments, at least one wire may comprise a super-elastic material, a shape-memory material, Nickel-Titanium (Nitinol®), platinum, cobalt chromium, stainless steel, tantalum, platinum iridium, ePTFE, a polymer, a metal, or any combination of these. In some embodiments, the expandable body comprises a straddling element extending between the first and second ends which will be described in detail in later sections.

In some embodiments, stents having least one loop extending from the first end may also include at least one constraining element configured to apply constraining force which holds the at least one loop in alignment with the longitudinal axis. The at least one constraining element releases the constraining force upon actuation by a releasing mechanism. Examples of such releasing mechanisms include a mechanical force, electrical energy, a chemical reaction, an electrochemical reaction, thermal energy, radiofrequency, ultrasonic energy, infrared radiation, change in pH, or any combination of these. In some embodiments, the at least one constraining element comprises a band or link. In other embodiments, the at least one constraining element comprises an expandable layer.

In a second aspect of the present invention, a method of positioning a stent is provided, wherein the stent comprises an radially expandable body having a first end, a second end and a longitudinal axis extending between the first and second ends, and at least one loop extending from the first end. The method includes mounting the stent on a first guidewire, wherein mounting comprises positioning a portion of the first guidewire within the at least one loop along the longitudinal axis causing at least the first end to transition toward the unexpanded state. In some embodiments, the method further includes advancing the stent over the first guidewire to a target location within a body lumen. The method may further include withdrawing the first guidewire from the at least one loop wherein such withdrawal allows the first end to expand within the body lumen. In preferred embodiments, the body lumen comprises a blood vessel. The target location may also include an aneurysm.

When the expandable body has a branched configuration and a third end, the method may further comprise mounting the stent on a second guidewire so that the first guidewire extends between the first and second ends, and the second guidewire extends between the first and third ends. The method may then further comprise advancing the stent simultaneously over the first and second guidewires to the target location. When the target location includes a branched portion of the body lumen and the first guidewire and the second guidewire are positioned in different branches of the branched portion of the body lumen, the method may further comprise advancing the stent so that the second and third ends are disposed within the different branches of the branched portion of the body lumen.

When the stent further comprises at least one constraining element configured to apply constraining force to assist in holding the at least one loop in alignment with the longitudinal axis, the method may further comprise releasing the constraining force by affecting the at least one constraining element by a releasing mechanism. The releasing mechanism typically comprises a mechanical force, electrical energy, a chemical reaction, an electrochemical reaction, thermal energy, radiofrequency, ultrasonic energy, infrared radiation, change in pH, or any combination of these.

In a third aspect of the present invention, a system for stenting a body lumen is provided comprising an radially expandable stent transitionable between an unexpanded state and expanded state, and at least one constraining element disposed around at least a portion of the stent which applies a constraining force to hold the at least a portion of the stent in the unexpanded state, wherein the at least one constraining element releases its constraining force upon actuation by a releasing mechanism.

In some embodiments, the releasing mechanism comprises a mechanical force which fractures the at least one constraining element. Thus, the system may further include a lead extending to the at least one constraining element, wherein the lead is configured to fracture the at least one constraining element by pulling, pushing, torquing, rotating or manipulating the lead. Alternatively or in addition, the system may further comprise an inflatable member disposed within the stent, wherein the inflatable member is configured to fracture the at least one constraining element upon inflation. A lead extending to the at least one constraining element may alternatively be configured to fracture the at least one constraining element by supplying electrical, thermal or radiofrequency energy to the lead.

In some embodiments, the at least one constraining element comprises an expandable layer which relaxes upon actuation by the releasing mechanism. When the expandable layer comprises a thermoplastic polymer, the releasing mechanism typically comprises heat. The system may also include at least one conductive coil disposed around the stent so that the heat is transferable from the at least one conductive coil to the expandable layer.

In other embodiments, the releasing mechanism comprises a chemical reaction. In still other embodiments, the releasing mechanism comprises thermal energy, radiofrequency, ultrasonic energy, infrared radiation, a change in pH or any combination of these.

The at least one constraining element typically extends around an exterior circumference of the stent. Alternatively or in addition, at least a portion of the at least one constraining element may extends through a wall of the stent. In any case, the at least one constraining element may include a stress region which is particularly responsive to the releasing mechanism.

In a fourth aspect of the present invention, a method is provided for treating a body lumen. The method includes positioning a stent within a target location of the body lumen, wherein the stent is transitionable between an unexpanded state and expanded state and includes at least one constraining element disposed around at least a portion of the stent which applies a constraining force to hold the stent in the unexpanded state, and actuating a releasing mechanism which causes the at least one constraining element to release the constraining force and allows the stent to transition toward the expanded state within the body lumen.

In some embodiments, actuating comprises fracturing the at least one constraining element. Fracturing may comprise applying a mechanical force. Alternatively or in addition, fracturing may comprise engaging a chemical reaction. In some embodiments, the method includes extending a lead to the at least one constraining element and wherein fracturing comprises supplying electrical, thermal or radiofrequency energy to the lead.

In some embodiments, the at least one constraining element comprises an expandable layer and actuating comprises causing the expandable layer to relax. Causing the expandable layer to relax may comprise heating the expandable layer. Relaxation of the expandable layer allows the stent to transition toward an expanded state.

In yet other embodiments, the releasing mechanism comprises thermal energy, radiofrequency, ultrasonic energy, infrared radiation, a change in pH or any combination of these.

In preferred embodiments, the body lumen comprises a blood vessel, and in some instances the target location includes an aneurysm. Alternatively or in addition, the target location may include a branched portion of the body lumen. In some embodiments, the stent has a branched configuration including a main branch and a side branch and the at least one constraining element comprises a first constraining element disposed around at least a portion of the main branch and a second constraining element disposed around at least a portion of the side branch. In such instances, the method further comprises actuating a releasing mechanism which causes the first and/or second constraining elements to release its constraining force. The method may further include positioning a first guidewire through the main branch and a second guidewire through the side branch and advancing the stent simultaneously over the first and second guidewires to the target location.

In some specific embodiments, a system for stenting a body lumen is provided comprising a radially expandable stent transitionable between an unexpanded state and expanded state; and at least one constraining element disposed around at least a portion of the stent which applies a constraining force to hold the at least a portion of the stent in the unexpanded state, wherein the at least one constraining element releases its constraining force upon actuation by a releasing mechanism.

Optionally, a system such as above is provided wherein the releasing mechanism comprises a mechanical force which fractures the at least one constraining element.

Optionally, any of the systems above further comprise a lead extending to the at least one constraining element, wherein the lead is configured to fracture the at least one constraining element by pulling, pushing, torqueing, rotating or manipulating the lead.

Optionally, any of the systems above further comprise an inflatable member disposed within the stent, wherein the inflatable member is configured to fracture the at least one constraining element upon inflation.

Optionally, any of the systems above further comprise a lead extending to the at least one constraining element, wherein the lead is configured to fracture the at least one constraining element supplying electrical, thermal or radiofrequency energy to the lead.

Optionally, any of the systems above are provided wherein the at least one constraining element comprises an expandable layer which relaxes upon actuation by the releasing mechanism. In at least some of these systems, the expandable layer comprises a thermoplastic polymer and the releasing mechanism comprises heat. And, at least some of these systems further comprise at least one conductive coil disposed around the stent so that the heat is transferable from the at least one conductive coil to the expandable layer.

Optionally, any of the systems above are provided wherein the releasing mechanism comprises a chemical reaction.

Optionally, any of the systems above are provided wherein the releasing mechanism comprises thermal energy, radiofrequency, ultrasonic energy, infrared radiation, a change in pH or any combination of these.

Optionally, any of the systems above are provided wherein the at least one constraining element includes a stress region particularly responsive to the releasing mechanism.

Optionally, any of the systems above are provided wherein the at least one constraining element extends around an exterior circumference of the stent.

Optionally, any of the systems above are provided wherein at least a portion of the at least one constraining element extends through a wall of the stent.

In some specific embodiments, a method of treating a body lumen is provided comprising positioning a stent within a target location of the body lumen, wherein the stent is transitionable between an unexpanded state and expanded state and includes at least one constraining element disposed around at least a portion of the stent which applies a constraining force to hold the stent in the unexpanded state; and actuating a releasing mechanism which causes the at least one constraining element to release the constraining force and allows the stent to transition toward the expanded state within the body lumen.

Optionally, a method such as above is provided wherein actuating comprises fracturing the at least one constraining element. In at least some of these methods, fracturing comprises applying a mechanical force. In at least some of these methods fracturing comprises engaging a chemical reaction.

Optionally, any of the above methods further comprise extending a lead to the at least one constraining element, wherein fracturing comprises supplying electrical, thermal or radiofrequency energy to the lead.

Optionally, any of the above methods are provided wherein the at least one constraining element comprises an expandable layer and wherein actuating comprises causing the expandable layer to relax. In at least some of these methods, causing the expandable layer to relax comprises heating the expandable layer.

Optionally, any of the above methods are provided wherein the releasing mechanism comprises thermal energy, radiofrequency, ultrasonic energy, infrared radiation, a change in pH or any combination of these.

Optionally, any of the above methods are provided wherein the body lumen comprises a blood vessel.

Optionally, any of the above methods are provided wherein the target location includes an aneurysm.

Optionally, any of the above methods are provided wherein the target location includes a branched portion of the body lumen.

Optionally, any of the above methods are provided wherein the stent has a branched configuration including a main branch and a side branch and wherein the at least one constraining element comprises a first constraining element disposed around at least a portion of the main branch and a second constraining element disposed around at least a portion of the side branch, the method further comprising actuating a releasing mechanism which causes the first and/or second constraining elements to release its constraining force. At least some of these methods further comprise positioning a first guidewire through the main branch and a second guidewire through the side branch and advancing the stent simultaneously over the first and second guidewires to the target location.

Other objects and advantages of the present invention will become apparent from the detailed description to follow, together with the accompanying drawings.

### BRIEF DESCRIPTION OF THE DRAWINGS

FIG. 1A illustrates an embodiment of a stent of the present invention having loops.

FIG. 1B illustrates the embodiment of FIG. 1A wherein the loops of the first end are drawn radially inwardly toward the longitudinal axis.

FIG. 2A-2B provide an end view of an embodiment of a stent wherein the loops and a portion of the frame are drawn radially inwardly toward an unexpanded state.

FIG. 3 illustrates the stent of FIG. 2B mounted on a guidewire.

FIG. 4 illustrates another embodiment of a stent of the present invention having loops.

FIGS. 5A-5B provide an end view of an embodiment of a stent wherein the loops and a portion of the frame are drawn radially inwardly toward an unexpanded state.

FIG. 6 illustrates the stent of FIG. 5B mounted on a guidewire.

FIG. 7 illustrates loops positioned so as to form a ring within the stent.

FIGS. 8A-8E illustrate embodiments of loops which extend substantially around or partially around a guidewire 30.

FIGS. 9-11 illustrate embodiments of stents of the present invention having a variety of shapes.

FIGS. 12A-12E illustrate a method of positioning a branched stent of the present invention into a bifurcated body lumen.

FIGS. 13A-13D illustrate a method of positioning a straddling stent of the present invention into a bifurcated body lumen having an aneurysm.

FIG. 14 illustrates the stent of FIG. 5B having bands holding the stent in an unexpanded state.

FIGS. 15A-15B illustrate a stent having links.

FIGS. 16A-16B illustrate an embodiment of a stent having constraining elements.

FIGS. 17A-17B illustrate an embodiment of a stent having constraining elements which are fracturable by a mechanical force.

FIGS. 18A-18B illustrate an embodiment of a stent having a constraining element in the form of an expandable layer.

FIGS. 19A-19B illustrate an embodiment of a stent having three expandable layers extending around the stent.

FIGS. 20A-20B illustrate an embodiment of a stent having multiple expandable layers.

FIGS. 21A-21D illustrate an embodiment of a stent having constraining elements comprising supports with expandable layers extending over the supports.

FIGS. 22A-22D illustrate a method of positioning a stent having constraining elements into a bifurcated body lumen having an aneurysm.

FIGS. 23A-23C illustrate an embodiment of a stent having a flexible line which passes through the loops in a manner so that applying tension to the line transitions the stent toward the unexpanded state.

### DETAILED DESCRIPTION OF THE INVENTION

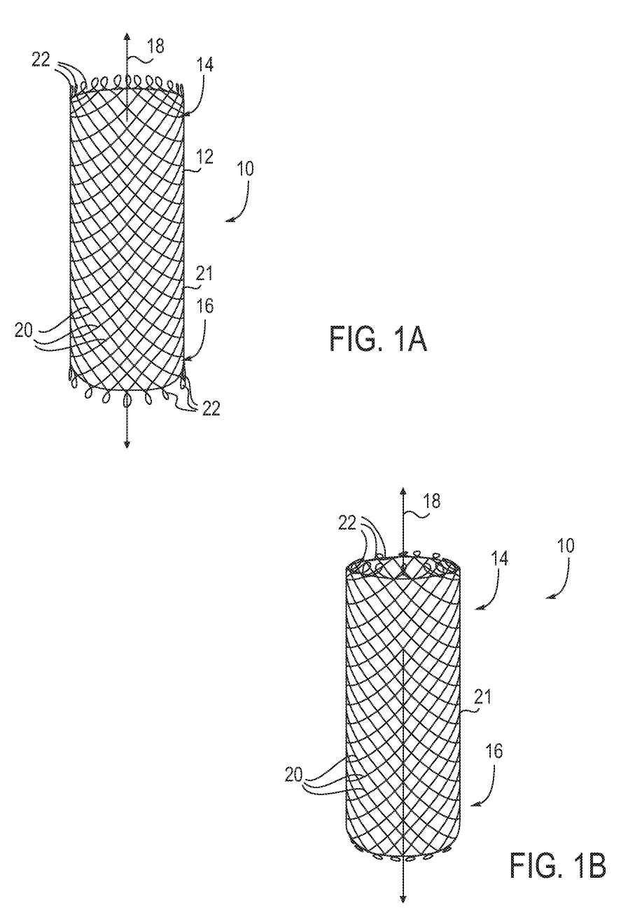

FIG. 1A illustrates an embodiment of a self-expanding stent 10 of the present invention. The stent 10 comprises an expandable body 12 having a generally tubular shape extending between a first end 14 and a second end 16 along a longitudinal axis 18. The expandable body 12 is transitionable between an unexpanded state, having a reduced cross-sectional diameter, and an expanded state having a greater cross-sectional diameter. In each of the described embodiments, the expandable body 12 is comprised of frame 21 formed from a plurality of wires 20 braided into a mesh or weave or formed by other methods, such as laser cutting, chemical etching or photo etching, to name a few. One or more portions of the frame 21 may be comprised of a superelastic material, a shape-memory material, Nickel-Titanium (Nitinol®), platinum, cobalt chromium, stainless steel, tantalum, gold, tungsten, platinum iridium, ePTFE, a polymer, a metal, Drawn Filled Tube (Nitinol® tube having a core volume filled with a radiopaque material such as gold, platinum, tantalum, platinum iridium, tungsten, etc.), an alloy, an alloy comprised of any of these, a combination of any of these or any other suitable material. When the stent 10 is comprised of a superelastic and/or shape-memory material, the stent self-expands due to the recoiling properties of the material.

The expandable body 12 includes at least one loop 22 extending from the expandable body 12. In this embodiment, a plurality of loops 22 are shown extending from both the first end 14 and the second end 16. The loops 22 may be integral with the frame 21 (as shown) or attached, coupled or joined with the frame 21. When the expandable body 12 is in the expanded state, the loops 22 are substantially parallel with the longitudinal axis 18, as shown. It may be appreciated that the longitudinal axis 18 may be concentric with the tubular shape of the expandable body 12, as shown, or may be offset within the expandable body 12. Thus, the longitudinal axis 18 may extend through the expandable body 12 at any distance from the walls of expandable body 12.

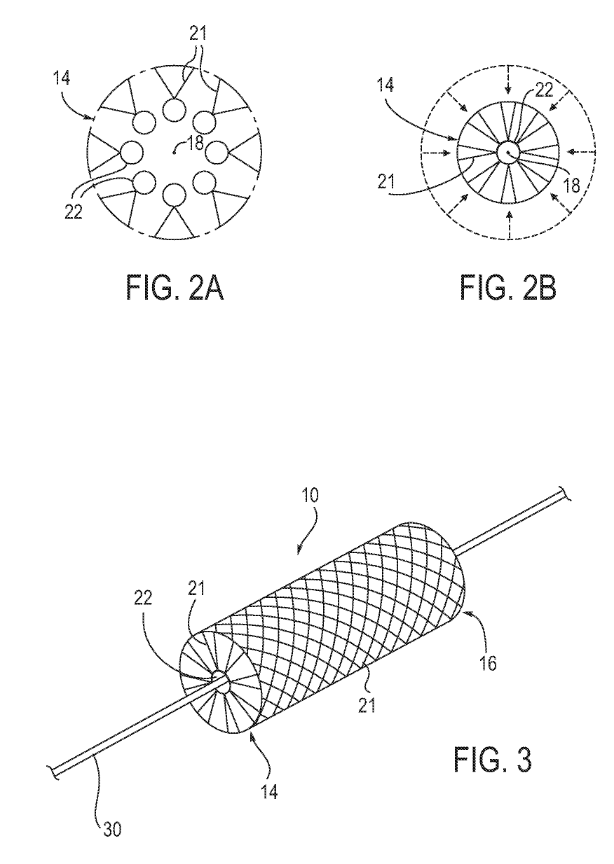

FIG. 1B illustrates the embodiment of FIG. 1A wherein the loops 22 of the first end 14 are drawn radially inwardly toward the longitudinal axis 18. It may be appreciated that the loops 22 and/or any portion of the frame 21 may be drawn radially inwardly at any angle in relation to the longitudinal axis 18, including perpendicular to the longitudinal axis. FIGS. 2A-2B provide an end view of the first end 14 as the loops 22 and a portion of the frame 21 are drawn radially inwardly. FIG. 2A illustrates the loops 22 and a portion of the frame 21 tilted toward the longitudinal axis while the expandable body 12 begins to transition toward the unexpanded state. FIG. 2B illustrates the loops 22 drawn together and aligned so that the longitudinal axis 18 passes through the loops 22. This in turn reduces the cross-sectional diameter of the expandable body 12, as indicated by arrows, further transitioning the body 12 toward the unexpanded state. The diameter of the stent in the unexpanded state may be a third of the diameter of the in the expanded state. And, it may be appreciated that the diameter ratio between the expanded and unexpanded states may vary with the number of loops 22 present and the extent of alignment, to name a few. It may also be appreciated that in some embodiments only some of the loops 22 are aligned with the longitudinal axis 18 causing transitioning toward the unexpanded state. Likewise, some of the loops 22 may be aligned with a first longitudinal axis 18′ and others of the loops 22 may be aligned with a second longitudinal axis 18″ wherein alignment with either or both axes 18′, 18″ causes transitioning toward the unexpanded state.

Referring to FIG. 3, a guidewire 30 may be passed through the aligned loops 22, thereby mounting the stent 10 on the guidewire. The guidewire 30 holds the loops 22 in the aligned orientation which in turn holds the stent 10 in an unexpanded state. The loops 22 present at the second end 16 may similarly be drawn radially inwardly so that the guidewire 30 passes through the loops 22 at both ends 14, 16 of the stent 10. The stent 10 may then be advanced along the guidewire 30 in the unexpanded state. Thus, the guidewire 30 may be positioned within any body lumen and the stent 10 advanced along the guidewire 30 to any suitable location within the body lumen, as will be described in later sections.

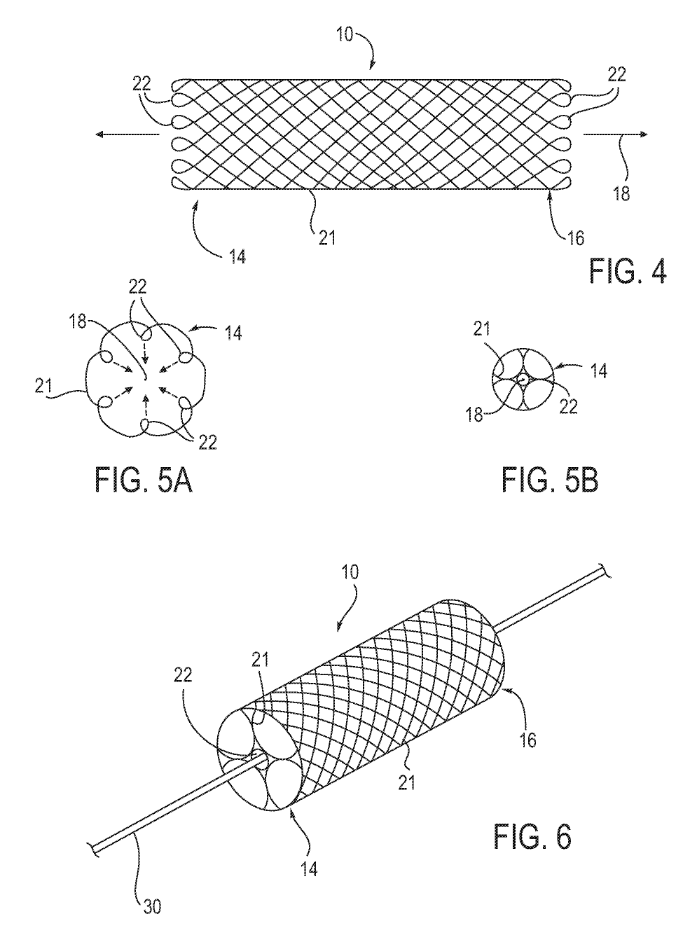

FIG. 4 illustrates a similar embodiment of a self-expanding stent 10 of the present invention. Again, the stent 10 comprises an expandable body 12 having a generally tubular shape extending between a first end 14 and a second end 16 along a longitudinal axis 18. The expandable body 12 is transitionable between an unexpanded state, having a reduced cross-sectional diameter, and an expanded state having a greater cross-sectional diameter. And, in this embodiment, the expandable body 12 is comprised of frame 21 formed from a plurality of wires 20 braided into a mesh or weave. The expandable body 12 includes at least one loop 22 extending from at least the first end 14 or the second end 16. In this embodiment, a plurality of loops 22 are shown extending from both the first end 14 and the second end 16.

FIG. 5A provides an end view of the first end 14 showing the loops 22 facing radially inwardly toward the longitudinal axis 18. The loops 22 are then drawn inwardly as indicated by arrows. FIG. 5B illustrates the loops 22 drawn together and aligned so that the longitudinal axis 18 passes through the loops 22. This in turn reduces the cross-sectional diameter of the expandable body 12, transitioning the body 12 toward the unexpanded state.

Referring to FIG. 6, a guidewire 30 may be passed through the aligned loops 22, thereby mounting the stent 10 on the guidewire. The guidewire 30 holds the loops 22 in the aligned orientation which in turn holds the stent 10 in an unexpanded state. The loops 22 present at the second end 16 may similarly be drawn radially inwardly so that the guidewire 30 passes through the loops 22 at both ends 14, 16 of the stent 10. The stent 10 may then be advanced along the guidewire 30 in the unexpanded state.

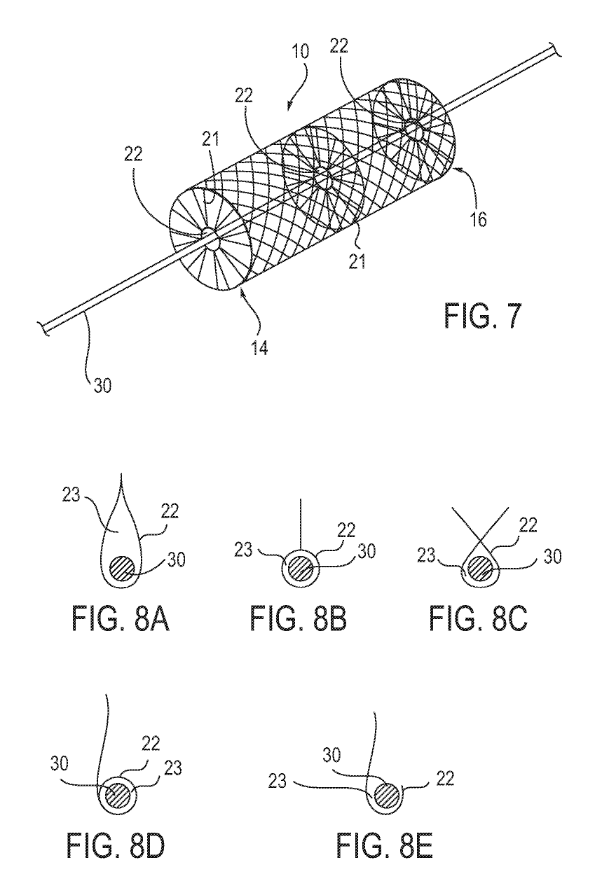

It may be appreciated that loops 22 may be present at any location along the stent 10, wherein the loops 22 are able to tilt so that their openings align with the longitudinal axis 18. For example, as illustrated in FIG. 7, the loops 22 may be positioned so as to form a ring within the stent 10 in a manner similar to loops 22 positioned at the first or second ends 14, 16. Thus, the loops 22 forming the ring would align with the longitudinal axis 18 in substantially the same location along the length of the axis 18. Alternatively or in addition, one or more loops 22 may be staggered along the length of the stent 10 so that the loops 22 align with the longitudinal axis 18 at various locations along its length. Desired locations for loops 22 may include mid-way along length of stent 10, near bifurcations of stent 10, and near the ends 14, 16, to name a few.

It may also be appreciated that loops 22 may have a variety of shapes, sizes or forms. Example embodiments of loops 22 of the present invention are illustrated in FIGS. 8A-8E. An end view of a guidewire 30 passing through an opening 23 of each of the loops 22 is shown. FIGS. 8A-8D illustrate embodiments of loops 22 which extend substantially around the guidewire 30. FIG. 8E illustrates an embodiment of a loop 22 which extends partially around the guidewire 30. It may be appreciated that the loop 22 may extend any distance around a guidewire 30 and/or may extend any number of times around a guidewire 30 (either at the same location or along the length of the guidewire 30). It may further be appreciated that the loop 22 may extend around or partially around the guidewire 30 in substantially the same plane or in more than one plane.

It may further be appreciated that the stent 10 may have any suitable form which allows alignment of the at least one loop 22 with the longitudinal axis 18. Thus, the expandable body 12 may be comprised of braids, coils, longitudinal struts, concentric struts, solid cylinders, or grided cylinders, to name a few. The solid cylinders may be comprised of elastic material or non-elastic material, wherein the non-elastic material is loosely fit in the unexpanded state to allow expansion. Likewise, the expandable body 12 may be laser cut, etched, covered, or uncovered to name a few. When covered, the expandable body 12 may be dipped in a liquid polymer so as to form a webbing across some or all of the openings of the frame 21. Or, the frame 21 may be covered by a jacket along some or all of the inside and/or outside surfaces of the frame 21. It may be appreciated that the embodiments described herein may be of any form, though they have been illustrated as uncovered for simplicity.

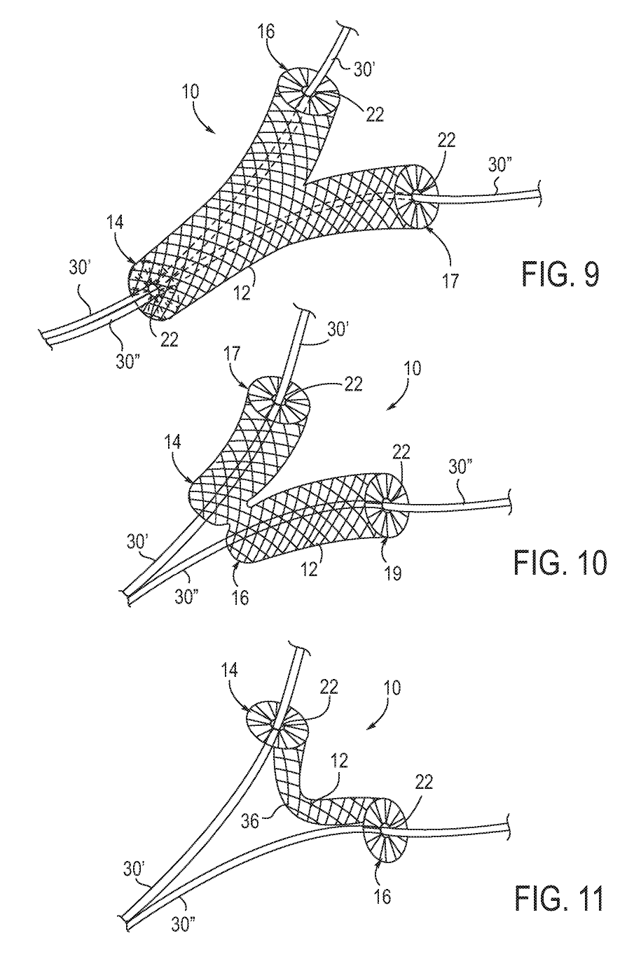

Further, the stent 10 may have a variety of shapes, examples of which are illustrated in FIGS. 9-11. FIG. 9 illustrates an embodiment of stent 10 having a bifurcated or branched shape. As shown, the expandable body 12 has a first end 14 and then branches into a second and third end 16, 17 which are substantially opposite to the first end 14. Each of the ends 14, 16, 17 include loops 22 which are drawn inwardly and align to allow passage of a guidewire therethrough. In this embodiment, a first guidewire 30′ passes through the first end 14 and the second end 16 and a second guidewire 30″ passes through the first end 14 and the third end 17. Thus, both guidewires 30′, 30″ pass through the first end 14. In this way, the stent 10 may be tracked over a pair of guidewires 30′, 30″ which are positioned within a branched or bifurcated body lumen, as will be described in later sections. It may be appreciated that it is not necessary for loops 22 to be present at all of ends 14, 16, 17, nor is it necessary for the guidewires to pass through loops 22 at each of ends 14, 16, 17. Typically, sufficient loops 22 are aligned and passed over the guidewire(s) to reduce the stent 10 to its unexpanded state.

FIG. 10 illustrates an embodiment of stent 10 having a connected shape. As shown, the expandable body 12 has a first end 14 and a second end 16 which are opposite to a third end 17 and fourth end 19. The first and second ends 16 are connected, as shown. Each of the ends 14, 16, 17, 19 include loops 22 which are drawn inwardly and align to allow passage of a guidewire therethrough. In this embodiment, a first guidewire 30′ passes through the first end 14 and the third end 17 and a second guidewire 30″ passes through the second end 16 and the fourth end 19. In this way, the stent 10 may be tracked over a pair of guidewires 30′, 30″ which are positioned within a branched or bifurcated body lumen, as will be described in later sections. Again, it may be appreciated that it is not necessary for loops 22 to be present at all of ends 14, 16, 17, 19 nor is it necessary for the guidewires to pass through loops 22 at each of ends 14, 16, 17, 19. Typically, sufficient loops 22 are aligned and passed over the guidewire(s) to reduce the stent 10 to its unexpanded state.

FIG. 11 illustrates an embodiment of stent 10 having a straddle shape. As shown, the expandable body 12 comprises a first end 14, a second end 16 and a straddling element 36 extending therebetween. In this embodiment, the straddling element 36 comprises an elongate sheet. Each of the ends 14, 16 include loops 22 which are drawn inwardly and align to allow passage of a guidewire therethrough. In this embodiment, a first guidewire 30′ passes through the first end 14 and a second guidewire 30″ passes through the second end 16. In this way, the stent 10 may be tracked over a pair of guidewires 30′, 30″ which are positioned within a branched or bifurcated body lumen, as will be described in later sections.

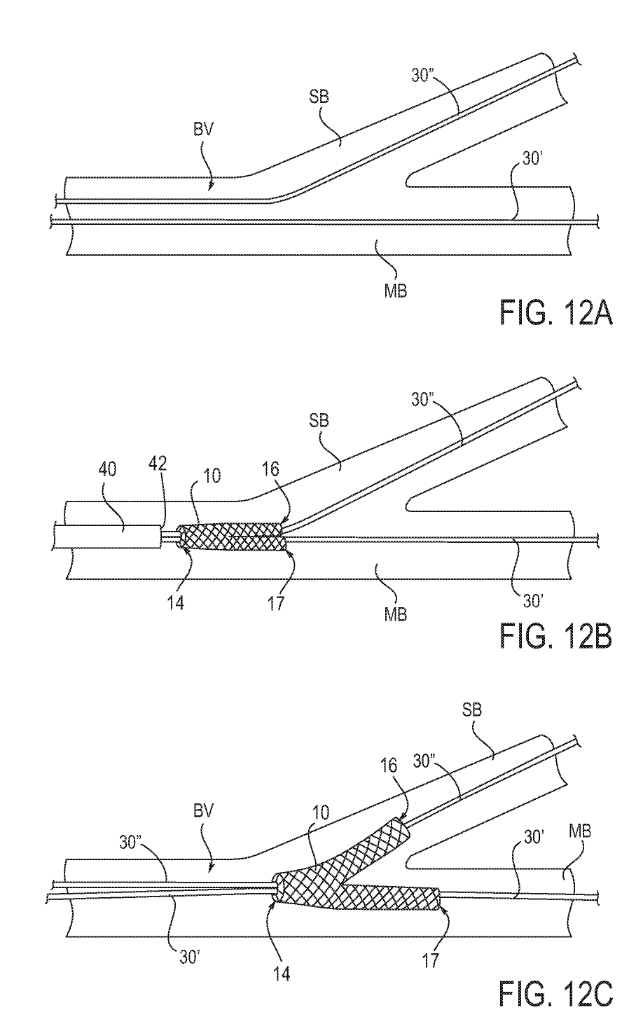

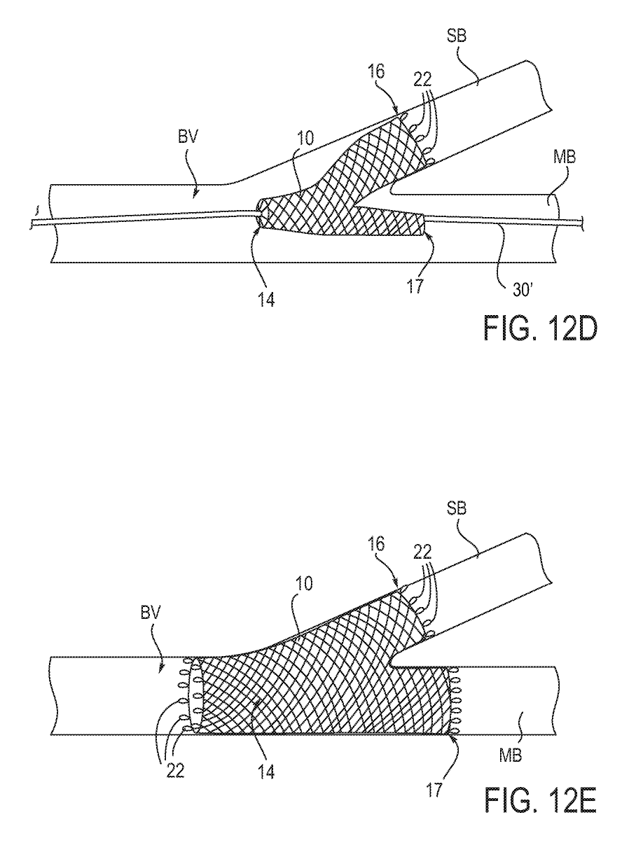

FIGS. 12A-12E illustrate a method of positioning a branched stent 10 of the present invention into a bifurcated body lumen. In this embodiment, the body lumen is a blood vessel BV having a main branch MB and a side branch SB. Referring to FIG. 12A, a first guidewire 30′ is positioned within the main branch MB and a second guidewire 30″ is positioned within the side branch SB. The branched stent 10 is loaded onto the guidewires 30′, 30″ in a manner described and illustrated in relation to FIG. 9. Such loading maintains the stent 10 in an unexpanded state.

Referring to FIG. 12B, the unexpanded stent 10 is then advanced over the guidewires 30′, 30″, such as by action of a pusher-release device 40. The pusher-release device 40 may have any suitable configuration so that the device 40 is advanceable over the guidewires 30′, 30″ and is able to push the stent 10 along the guidewires 30′, 30″. Thus, the pusher-release device 40 may simply comprise a tube having a pushing face 42 which is mateable against the stent 10. Or, the pusher-release device 40 may be joinable with the stent 10 to allow advancement and retraction of the stent 10 along the guidewires 30′, 30″, wherein the device 40 is releasable from the stent 10 when the stent 10 is desirably placed. As shown, the guidewires 30′, 30″ are substantially parallel within the main branch MB. Due to the flexibility of the stent 10, the second end 16 and third end 17 of the stent 10 are positioned substantially parallel as well.

Referring to FIG. 12C, the stent 10 is advanced so that the second end 16 extends into the side branch SB and the first and third ends 14, 17 remain in the main branch MB as shown. Once the stent 10 is desirably positioned within the bifurcation of the blood vessel BV, the guidewires 30′, 30″ are removed. The guidewires 30′, 30″ may be removed simultaneously, in series, or in any suitable combination of movements. FIG. 12D shows the first guidewire 30′ removed while the second guidewire 30″ remains in place. Removal of the first guidewire 30′ releases the associated loops 22 and allows the second end 16 to self-expand within the side branch SB. However, the first end 14 and the third end 17 remain in the unexpanded state maintained by the second guidewire 30″. FIG. 12E shows the second guidewire 30″ removed which releases the associated loops 22 and allows the first end 14 and third end 17 to self-expand within the main branch MB.

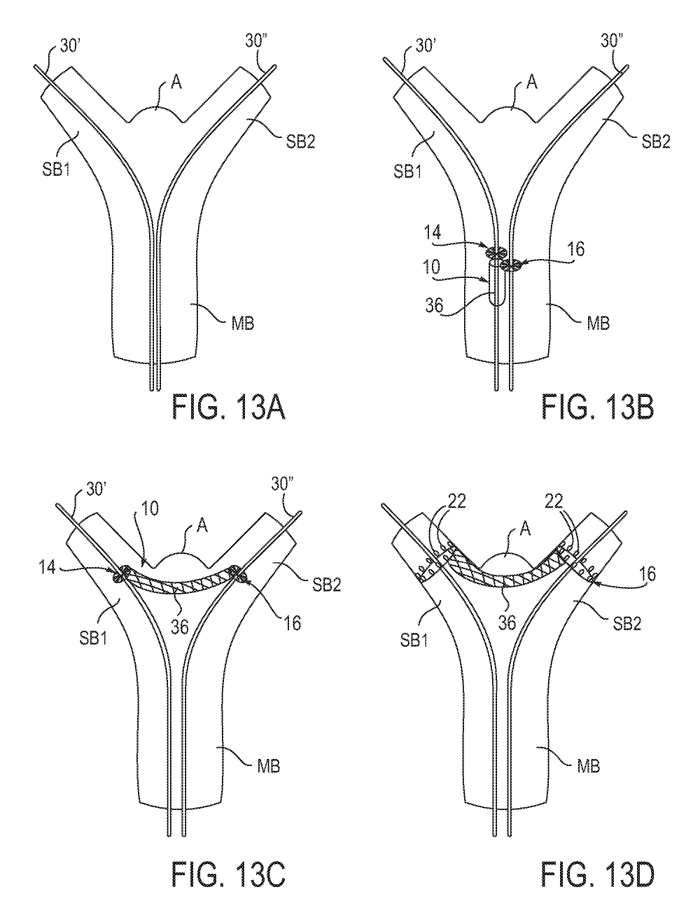

FIGS. 13A-13D illustrate a method of positioning a straddling stent 10 of the present invention into a bifurcated body lumen having an aneurysm. In this embodiment, the body lumen comprises a cerebral blood vessel BV having a main branch MB, a first side branch SB1, a second side branch SB2, and an aneurysm A therebetween. Such location of the aneurysm is a relatively common challenge when treating cerebral aneurysms. Typically, it is desired to exclude the aneurysm, such as by creating an artificial lumen wall by covering the aneurysm with a straddling element 36 such as described and illustrated in relation to FIG. 11.

Referring to FIG. 13A, a first guidewire 30′ is positioned within the main branch MB and the first side branch SB1 and a second guidewire 30″ is positioned within the main branch MB and the second side branch SB2. The straddling stent 10 is loaded onto the guidewires 30′, 30″ in a manner described and illustrated in relation to FIG. 11. Such loading maintains the stent 10 in an unexpanded state.

Referring to FIG. 13B, the unexpanded stent 10 is then advanced over the guidewires 30′, 30″, such as by action of a pusher-release device 40 (not shown). The pusher-release device 40 may have any suitable configuration so that the device 40 is advanceable over the guidewires 30′, 30″ and is able to push the stent 10 along the guidewires 30′, 30″. The device 14 may push against first end 14 and second end 16 as the straddling element 36 is maintained therebetween.

Referring to FIG. 13C, the stent 10 is advanced so that the first end 14 extends into the first side branch SB1 and the second end 16 extends into the second side branch SB2. The ends 14, 16 are sufficiently advanced to desirably position the straddling element 36 over the aneurysm A. Advancement of the ends 14, 16 may be achieved with the use of a single pusher-release device 40 pushing individually on the ends 14, 16, two separate pusher-release devices 40 each pushing on an end 14, 16 or a specially designed device 40 having a branched end for pushing on the ends 14, 16. Once the stent 10 is desirably positioned, the guidewires 30′, 30″ are removed. The guidewires 30′, 30″ may be removed simultaneously, in series, or in any suitable combination of movements. FIG. 13D shows the guidewires 30′, 30″ removed, releasing the associated loops 22 which allow the first end 14 to self expand within the first side branch SB1 and the second end 16 to self expand within the second side branch SB2.

It may be appreciated that upon release of a loop 22, by withdrawal of a guidewire 30 or other structure, the loop 22 will recoil away from the longitudinal axis 18 to a relaxed position. In this relaxed position, the loop 22 may be tilted toward the longitudinal axis 18, substantially aligned with the tubular walls of the stent 10 or tilted outwardly from the walls of the stent 10. Outward tilting of the loops 22 may assist in anchoring the stent 10 within a vessel.

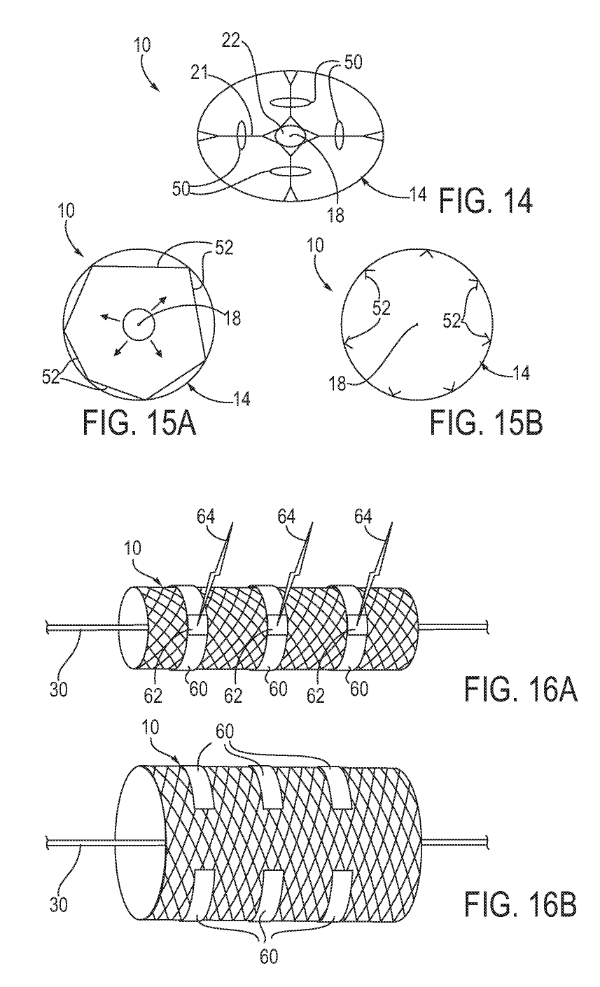

The force required to remove one or more guidewires from the loops of the stents may vary depending on a variety of factors including geometry of the loops, number of loops, geometry of the stent, stent material (e.g. thickness of the wire of the frame), presence of lubricious coatings, etc. In some embodiments, removal force may be reduced with the use of constraining elements, such as bands, which assist in holding the stent in the unexpanded state. For example, FIG. 14 illustrates the stent 10 of FIG. 5B having bands 50 holding the stent in the unexpanded state. Here, the first end 14 is shown having the loops 22 facing radially inwardly toward the longitudinal axis 18. Bands 50 are formed or wrapped around portions of the loops 22 and/or frame 21. In this embodiment, the bands 50 hold together portions of the frame 21 which are directed radially inward toward the longitudinal axis 18. The stent 10 is free to move along a guidewire, etc., as described above while the bands 50 are in place. Once the stent 10 has been desirably positioned, the guidewire(s) may be removed with ease while the bands 50 hold the stent 10 in the unexpanded state. The bands 50 are then broken or fractured which allows the stent to self-expand. It may be appreciated that the bands 50 may be fractured at any time, before, during or after removal of the guidewire(s) as desired.

The bands 50 may be broken using any of a variety of releasing mechanisms, particularly including the application of a mechanical force, electrical energy, a chemical reaction, an electrochemical reaction, thermal energy, radiofrequency, ultrasonic energy, infrared radiation, change in pH, etc. The bands 50 may be comprised of any suitable material which is responsive to one or more releasing mechanisms. Typically the bands 50 are relatively non-extendable to assist in holding the stent in the unexpanded state. The bands 50 may also include a predetermined stress region that is designed to fail under the application of the releasing mechanism.

Alternatively or in addition to the bands 50, links 52 may be present within the stent 10, as illustrated in FIGS. 15A-15B. FIG. 15A illustrates the stent 10 of FIG. 14 having links 52 holding the stent in the unexpanded state. Here, the first end 14 is shown having the loops 22 facing radially inwardly toward the longitudinal axis 18, however portions of the frame 21 directed radially inwardly are not shown for clarity. Once the stent 10 has been desirably positioned, the guidewire(s) may be removed with ease while the links 52 hold the stent 10 in the unexpanded state. The links 52 are then broken or fractured which allows the stent to self-expand. The links 52 may be fractured using any of a variety of releasing mechanisms, particularly including the application of a mechanical force, electrical energy, a chemical reaction, an electrochemical reaction, thermal energy, radiofrequency, ultrasonic energy, infrared radiation, change in pH, etc. The links 52 may be comprised of any suitable material which is responsive to one or more releasing mechanisms. It may be appreciated that the links 52 may be fractured at any time, before, during or after removal of the guidewire(s) as desired. FIG. 15B illustrates the stent 10 of FIG. 15A in the expanded state wherein the links 52 are shown fractured. It may be appreciated that the links 52 have been exaggerated in size for illustration purposes.

The above described bands 50 and links 52 are typically used in conjunction with stents 10 having loop 22 features, wherein the bands 50 or links 52 assist in holding the loops 22 in alignment which in turn holds the stent 10 in an unexpanded state. In other embodiments, constraining elements 60 are provided which hold the stent 10 in an unexpanded state. Such constraining elements 60 may be comprised of the same or similar material as bands 50 and/or links 52 and may be fractured by the same or similar releasing mechanisms, particularly including the application of a mechanical force, electrical energy, a chemical reaction, an electrochemical reaction, thermal energy, radiofrequency, ultrasonic energy, infrared radiation, change in pH, etc. However, the constraining elements 60 may be used with any stent design, including conventional stents. When used with conventional self-expanding stents, the constraining elements 60 eliminate the need for a sheath to hold the self-expanding stent in an unexpanded state, thereby reducing the profile of the stent during delivery. It may be appreciated that the constraining elements 60 of the present invention may also be used with the stents 10 of the present invention having loops 22.

FIGS. 16A-16B illustrate an embodiment of a stent 10 having constraining elements 60. Here, three constraining elements 60 are present and wrap around the exterior perimeter of the stent. It may be appreciated that the constraining elements 60 may alternatively weave through portions of the stent 10, wrap around the interior of the stent in an adhered fashion, and/or wrap to form a coil shape. Or the constraining elements 60 may have other forms including hooks. As mentioned, the constraining elements 60 hold the stent 10 in its unexpanded state negating the need for a conventional external sheath. The stent 10 may be advanced over a guidewire 30 in a manner similar to the stents 10 described above having loops 22, or the stent 10 may be mounted on a catheter or other delivery device which is advanced over the guidewire 30. In either case, the lack of external sheath allows delivery of a variety of different shaped stents, including branched, connected or other configurations.

FIG. 16A illustrates an embodiment wherein each constraining element 60 includes a stress region 62 which is configured to fracture when subjected to a releasing mechanism, as indicated by activation bolts 64. It may be appreciated that each constraining element 60 may include multiple stress regions 62 arranged in any configuration or the entire constraining element 60 may act as a stress region 62 responding to the releasing mechanism. Upon fracturing of the constraining elements 60, the stent 10 is released from the unexpanded or constrained state and allowed to self-expand, as illustrated in FIG. 16B.

In some embodiments, the releasing mechanism comprises a chemical reaction or process. In such embodiments, the stress region 62 may comprise a sacrificial element which dissolves, corrodes or degrades when it reacts with a particular chemical substance. The chemical substance may be provided by the body environment or may be externally provided by the practitioner. Once the sacrificial element is weakened or consumed, the stent 10 is released from constraining forces and allowed to self-expand while the non-sacrificial element or remainder of the constraining element is still present. Some chemical processes create thermal energy which is used to melt the stress region 62 causing fracture. For example, the stress region 62 may react with a catalyst which is provided by the body environment or externally provided by the practitioner. In other embodiments, the chemical reaction simply weakens the constraining element 60 so that the constraining element 60 may be fractured by an alternate force, such as a mechanical force.

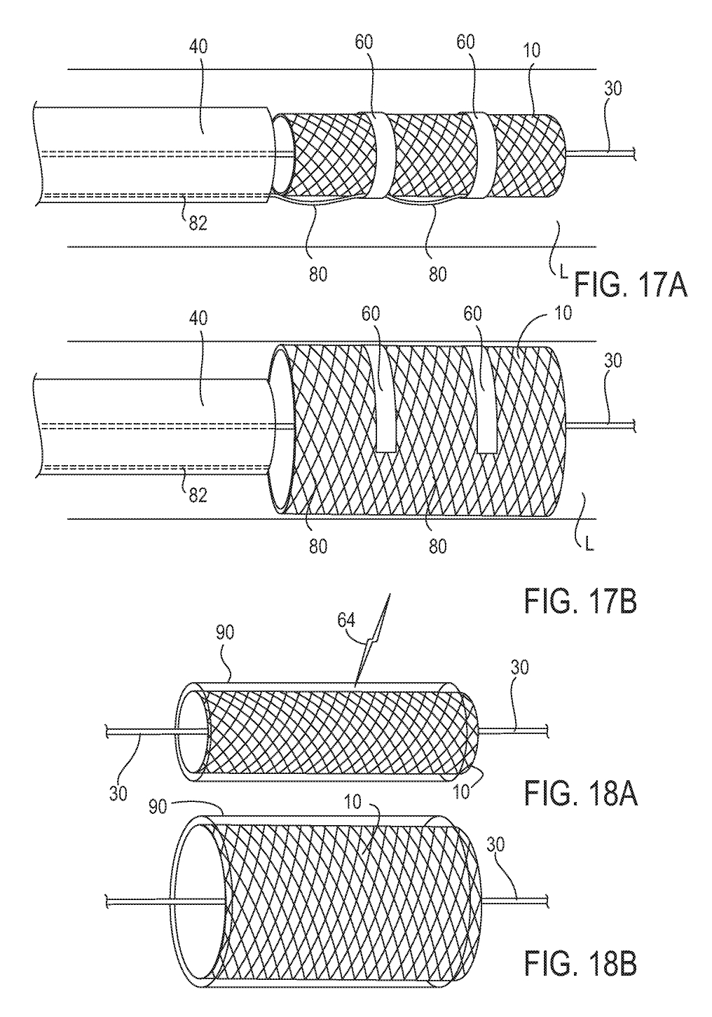

In some embodiments, the releasing mechanism comprises a mechanical force. For example, FIGS. 17A-17B illustrate a stent 10 having constraining elements 60 which are fracturable by a mechanical force. The stent 10 is shown advanced over a guidewire 30 within a body lumen L. Referring to FIG. 17A, the stent 10 is advanced by a pusher-release device 40 having a lead 80 which extends to the constraining elements 60. In this embodiment, the lead 80 comprises a wire which passes through a lead lumen 82 in the device 40 and extends to each of the constraining elements 60 in series. Alternatively, the lead 80 may comprise a suture, strand, thread, filament, rod or other suitable element. When the lead 80 is pulled, pushed, torqued, rotated or otherwise manipulated, the constraining elements 60 fracture allowing the stent 10 to self expand, as illustrated in FIG. 17B. It may be appreciated that numerous leads 80 may be present, each extending to a separate constraining element 60 allowing the constraining elements 60 to be fractured independently. Or any other lead 80 configuration may be present. The lead 80 may then be retracted through the device 40 or the device 40 may simply be removed together with the lead 80. The lead 80 may also function to connect or join the stent 10 with the pusher-release device 40 during delivery. This allows the device 40 to advance the stent 10 by pushing and retract the stent 10 by pulling the lead 80 to adjust the position of the stent 10.

Other mechanical forces which are able to fracture the constraining element 60 may be applied with the use of an expandable member, such as an inflatable balloon. The stent 10 may be mounted on a balloon of a delivery catheter so that inflation of the balloon applies outward radial force to the stent 10, fracturing the constraining elements 60. Upon fracture of the constraining elements 60, the stent 10 is allowed to self-expand. Thus, the balloon simply provides the mechanical force to release the stent from constraint. Consequently, a lower profile balloon may be used than with balloon-expandable stents which require the balloon to expand to the full desired inner diameter of the stent in the expanded state.

In some embodiments, the releasing mechanism comprises an electrical force. For example, the leads 80 as illustrated in FIG. 17A may be capable of conducting current to the constraining elements 60. When electrical energy is applied to the lead 80 the constraining elements 60 fracture allowing the stent 10 to self expand, as illustrated in FIG. 17B. Or, the leads 80 as illustrated in FIG. 17A may be capable of conducting heat to the constraining elements 60 causing the constraining element 60 to fracture by thermal force. Again, it may be appreciated that numerous leads 80 may be present, each extending to a separate constraining element 60 allowing the constraining elements 60 to be fractured independently. Or any other lead 80 configuration may be present. Further, the leads 80 may be used to provide any combination of mechanical, electrical and thermal forces. In addition, it may be appreciated that the constraining elements 60 may be fractured by any combination of releasing mechanisms described herein.

FIGS. 18A-18B illustrate an embodiment of a stent 10 having a constraining element 60 in the form of an expandable layer 90. Referring to FIG. 18A, the layer 90 is comprised of a material which may be softened or relaxed upon activation by a releasing mechanism, as indicated by activation bolt 64. Such relaxation releases the constraining force on the stent 10 allowing the stent to self-expand, as shown in FIG. 18B. The layer 90 thus expands as well. The layer 90 may be in the form of an external covering, coating or sleeve or may be formed within the walls of the stent 10, such as a webbing between wires of a frame. Similarly, the layer 90 may be a coating or covering adhered to the interior of the stent 10. Example materials include thermoplastic polymers, such as fluorinated ethylene propylene (FEP), nylon, polyester, polyurethane, low density polyethylene (LDPE), Pebax, polyethylene (PE). In such instances, the stent 10 may be heated (such as by electrical or radiofrequency energy) via a conduction wire inside of a delivery catheter used to deliver the stent. The heated stent 10 then heats the thermoplastic polymer causing the polymer to soften and reform. This releases the constraining force on the stent 10 allowing the stent 10 to self-expand.

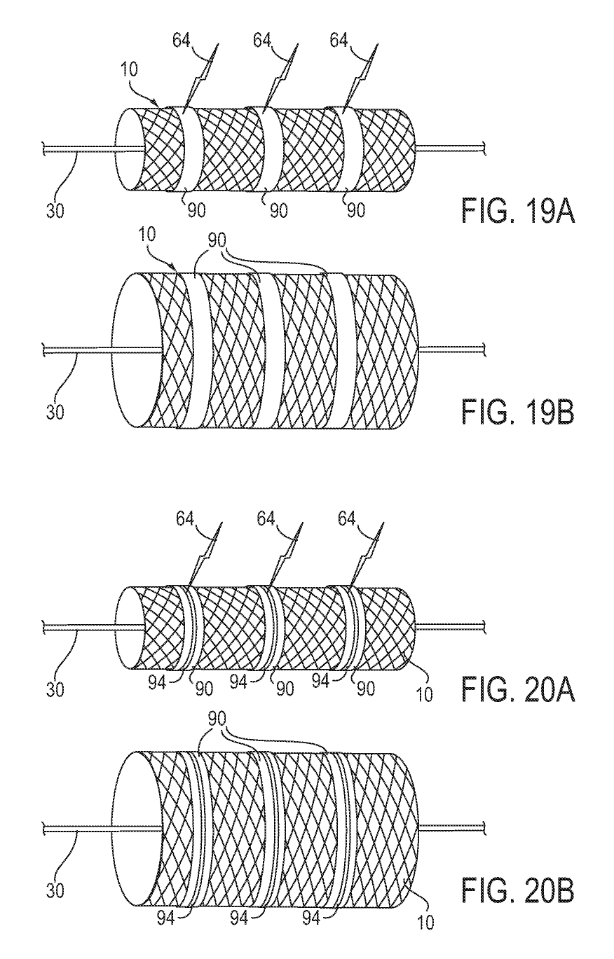

The layer 90 may extend over the entire stent 10, as illustrated in FIGS. 18A-18B, or may cover portions of the stent 10. FIGS. 19A-19B illustrate three expandable layers 90 extending around the stent 10. Each of the layers 90 be softened or relaxed upon activation by a releasing mechanism, as indicated by activation bolts 64. However, each layer 90 may be activated by a different type of releasing mechanism. Or, each layer 90 may be activated at a different threshold by the same type of releasing mechanism, such as at different temperatures. Or, each layer 90 may react in different manners to the same or different type of releasing mechanism. For example, one layer may soften or relax at a faster rate than another layer present on the stent 10. In any case, such relaxation releases the constraining force on the stent 10 allowing the stent to self-expand, as shown in FIG. 19B.

FIGS. 20A-20B illustrate a similar embodiment of a stent 10 having a constraining element 60 in the form of expandable layers 90. Referring to FIG. 20A, the layers 90 are comprised of a material which may be softened or relaxed upon heating as indicated by activation bolt 64. In this embodiment, heating of the layers 90 is assisted by the presence of conductive coils 94, such as thin filaments. Each coil 94 extends around the stent 10 and is in contact with a layer 90. Typically, the layer 90 covers or encases the coil 94, holding the coil 94 in place. The coil 94 is then heated via electrical or radiofrequency energy, melting or relaxing the layer 90. Such relaxation releases the constraining force on the stent 10 allowing the stent to self-expand, as shown in FIG. 20B. Thus, the layer 90 and coil 94 expand as well.

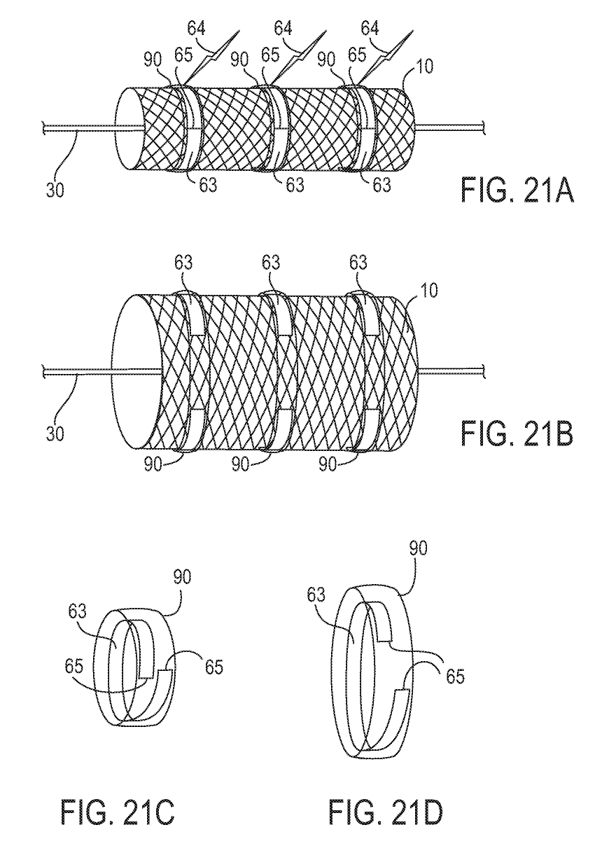

FIGS. 21A-21D illustrate an embodiment of a stent 10 having constraining elements 60 comprising supports 63 with expandable layers 90 extending over the supports 63. Together, the supports 63 and expandable layers 90 hold the stent 10 in its unexpanded state negating the need for a conventional external sheath. In this embodiment, the supports 63 crimp around the stent 10 and the expandable layers 90 hold the supports 90 in their crimped arrangement. For example, as shown in FIG. 21A the expandable layers 90 hold the edges 65 of the supports 90 together. When a releasing mechanism is provided, as indicated by activation bolts 64, the layers 90 soften or relax. Such relaxation allows the supports 63 to release their crimping action which in turn allows the stent 10 to self-expand, as illustrated in FIG. 21B. It may be appreciated that the supports 63 may have any width, thickness, length or configuration. For example, FIGS. 21C-21D illustrate an embodiment of the constraining elements 60 wherein the support 63 extends around the stent 10 (not shown) so that its edges 65 overlap and the expandable layer 90 holds the edges 65 in place. When a releasing mechanism is provided, the layer 90 relaxes. Such relaxation allows the supports 63 to release their crimping action, as illustrated in FIG. 21D.

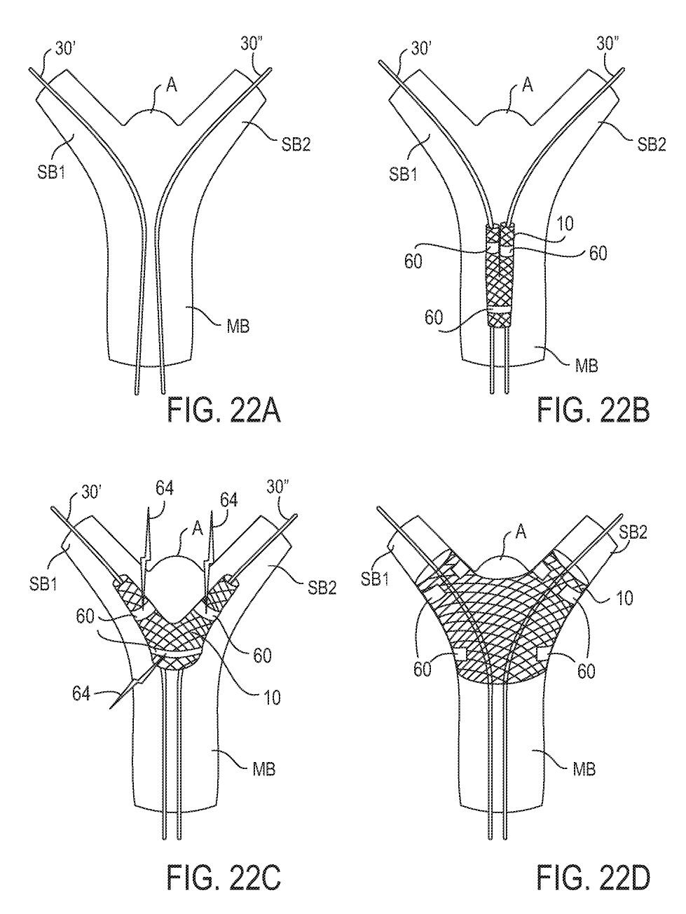

FIGS. 22A-22D illustrate a method of positioning a stent 10 having constraining elements 60 into a bifurcated body lumen having an aneurysm. Such methods are similar to those described in relation to FIGS. 13A-13D wherein the stent 10 may be advanced directly over guidewires. However, in this example the stent 10 is held in an unexpanded state by the constraining elements 60 rather than by loops of the stent. Thus, the stent 10 of FIGS. 13A-13D may be any conventional stent lacking such loops.

In this embodiment, the body lumen comprises a cerebral blood vessel BV having a main branch MB, a first side branch SB1, a second side branch SB2, and an aneurysm A therebetween. Referring to FIG. 22A, a first guidewire 30′ is positioned within the main branch MB and the first side branch SB1 and a second guidewire 30″ is positioned within the main branch MB and the second side branch SB2, both by the Seldinger technique or suitable methodologies. The stent 10 is loaded onto the guidewires 30′, 30″, such loading maintains the stent 10 in an unexpanded state.

Referring to FIG. 22B, the unexpanded stent 10 is then advanced over the guidewires 30′, 30″, such as by action of a pusher-release device 40 (not shown). The pusher-release device 40 may have any suitable configuration so that the device 40 is advanceable over the guidewires 30′, 30″ and is able to push the stent 10 along the guidewires 30′, 30″. Referring to FIG. 22C, the stent 10 is advanced to desirably position the stent 10 over the aneurysm A. Once the stent 10 is desirably positioned, the constraining elements 60 are activated by a releasing mechanism, as indicated by activation bolts 64, as illustrated in FIG. 22C. Upon activation or fracturing of the constraining elements 60, the stent 10 is released from the unexpanded or constrained state and allowed to self-expand, as illustrated in FIG. 22D. The guidewires 30′, 30″ may then be removed.

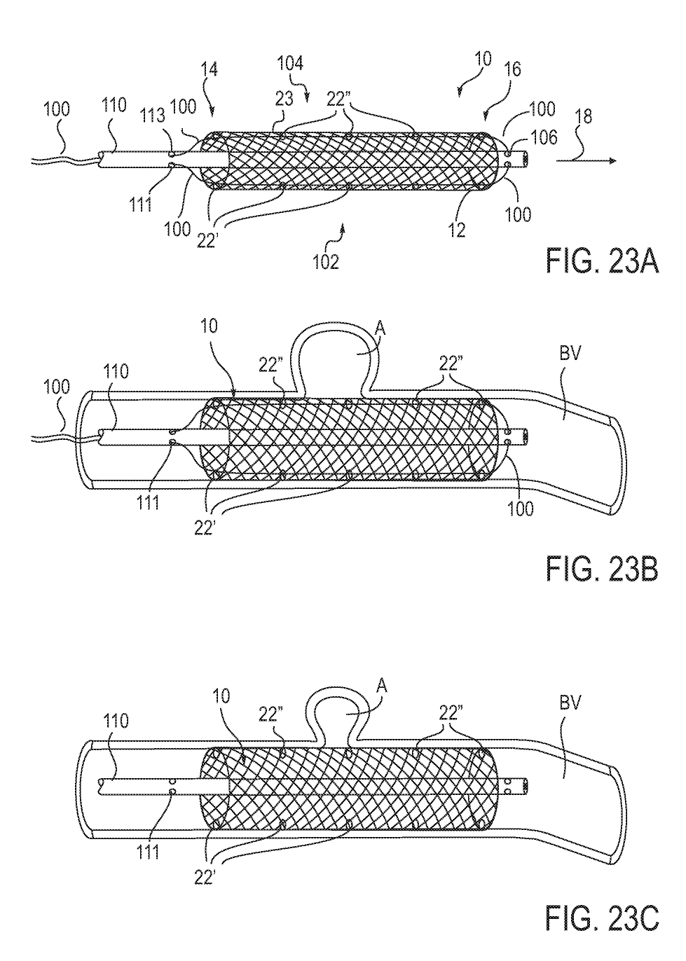

FIGS. 23A-23C illustrate another embodiment of a stent 10 of the present invention. Again, the stent 10 comprises an expandable body 12 having a generally tubular shape extending between a first end 14 and a second end 16 along a longitudinal axis 18. The expandable body 12 is transitionable between an unexpanded state, having a reduced cross-sectional diameter, and an expanded state having a greater cross-sectional diameter. And, in this embodiment, the expandable body 12 is self expanding and is comprised of frame 21 formed from a plurality of wires 20 braided into a mesh or weave. The expandable body 12 includes at least one loop 22 having an opening 23, the at least one loop extending from at least the first end 14 or the second end 16 and optionally at a variety of locations along the stent 10 as shown.

As shown in FIG. 23A, the stent 10 is loaded on a delivery device 110 for delivery to a blood vessel. The delivery device 110 may have any suitable shape, and includes an elongate distal tip 108 upon which the stent 10 is loaded. The loops 22 are configured so that an elongate structure, such as a flexible line 100, is passable through the openings 23 of the loops 22. The flexible line 100 passes through the loops 22 in a manner so that applying tension to the line 100 transitions the stent 10 toward an unexpanded state. The flexible line 100 may have any suitable form such as a suture, thread, fiber, filament, strand, wire, or ribbon, to name a few. In this example, the flexible line 100 extends through the delivery device 110 and exits through an aperture 111 near the first end 14 of the stent 10. The flexible line 100 then passes through loops 22′ that are disposed along a side 102 of the stent 10. The flexible line 100 then passes through the distal tip 108 of the delivery device 110 via apertures 106 near the second end 16 to an opposite side 104 of the stent 10, wherein the line 100 then passes through loops 22″ that are disposed along the opposite side 104 of the stent 10. The line 100 then passes back into the delivery device 110 through an aperture 113 and extends proximally. When force is applied to the line 100 in a proximal direction along both sides 102, 104 of the stent 10, the line 100 is held by the delivery device 110 via apertures 106 and the portions of the line 100 passing through the loops 22 are drawn radially inwardly toward the delivery device 110. This in turn draws the sides 102, 104 of the stent 10 radially inwardly collapsing the stent 10 toward the unexpanded state.

FIG. 23B illustrates the stent 10 positioned within a blood vessel BV so that the stent 10 straddles an aneurysm A. Release of tension on the flexible line 100 allows the self-expanding stent 10 to transition toward the expanded state as shown. The flexible line 100 may then be removed by pulling the line 100 proximally along one of the sides 102, 104 (e.g. the line 100 may be pulled proximally through aperture 111 until the entire line 100 is removed from the stent 10). FIG. 23C illustrates the stent 10 positioned within the blood vessel BV wherein the line 100 has been removed. The delivery device 110 may then be removed and the stent 10 left in place.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

All publications and patent applications cited in this specification are herein incorporated by reference as if each individual publication or patent application were specifically and individually indicated to be incorporated by reference.

What is claimed is:

1. A stent for positioning within a body lumen comprising:
a radially expandable body having a first end, a second end and a longitudinal axis extending between the first and second ends, the expandable body transitionable between an unexpanded state and an expanded state; and
at least one loop having an opening extending from the first end, wherein alignment of the opening of the at least one loop with the longitudinal axis transitions at least the first end toward the unexpanded state.

2. A stent as in claim 1, wherein the at least one loop is configured for passage of at least one guidewire therethrough when its opening is positioned in alignment with the longitudinal axis.

3. A stent as in claim 2, wherein the expandable body is advanceable directly over the at least one guidewire.

4. A stent as in claim 1, wherein the at least one loop comprises a plurality of loops extending around a circumference of the first end.

5. A stent as in claim 1, further comprising at least one loop having an opening extending from the second end, wherein alignment of the opening of the at least one loop extending from the second end with the longitudinal axis transitions the second end toward the unexpanded state.

6. A stent as in claim 1, wherein the expandable body further comprises a third end and another longitudinal axis extending between the first and third ends, and further comprising at least one loop having an opening extending from the third end, wherein alignment of the opening of the at least one loop extending from the third end with the other longitudinal axis transitions the third end toward the unexpanded state.

7. A stent as in claim 6, wherein the at least one loop of the third end is configured for passage of at least one guidewire therethrough when its opening is positioned in alignment with the other longitudinal axis.

8. A stent as in claim 7, wherein the expandable body is simultaneously advanceable directly over a first guidewire passed through the first and second ends and a second guidewire passed through the first and third ends.

9. A stent as in claim 1, wherein the expandable body comprises a frame formed from a plurality of wires.

10. A stent as in claim 1, wherein the expandable body comprises a frame formed from a super-elastic material, a shape-memory material, Nickel-Titanium (Nitinol®), platinum, cobalt chromium, stainless steel, tantalum, gold, tungsten, platinum iridium, ePTFE, a polymer, a metal, a Nitinol® tube having a core volume filled with a radiopaque material, an alloy, an alloy comprised of any combination of these or any combination of these.

11. A stent as in claim 1, wherein the expandable body comprises a straddling element extending between the first and second ends.

12. A stent as in claim 1, further comprising at least one constraining element configured to apply constraining force which holds the opening of the at least one loop in alignment with the longitudinal axis, wherein the at least one constraining element releases the constraining force upon actuation by a releasing mechanism.

13. A stent as in claim 12, wherein the at least one releasing mechanism comprises a mechanical force, electrical energy, a chemical reaction, an electrochemical reaction, thermal energy, radiofrequency, ultrasonic energy, infrared radiation, change in pH, or any combination of these.

14. A stent as in claim 12, wherein the at least one constraining element comprises a band or link.

15. A stent as in claim 12, wherein the at least one constraining element comprises an expandable layer.

16. A method of positioning a stent, wherein the stent comprises an radially expandable body having a first end, a second end and a longitudinal axis extending between the first and second ends, and at least one loop extending from the first end, the method comprising:
mounting the stent on a first guidewire, wherein mounting comprises positioning a portion of the first guidewire within the at least one loop along the longitudinal axis causing at least the first end to transition toward the unexpanded state.

17. A method as in claim 16, further comprising advancing the stent over the first guidewire to a target location within a body lumen.

18. The method of claim 17, further comprising withdrawing the first guidewire from the at least one loop wherein such withdrawal allows the first end to expand within the body lumen.

19. A method as in claim 17, wherein the body lumen comprises a blood vessel.

20. A method as in claim 17, wherein the target location includes an aneurysm.

21. A method as in claim 16, wherein the expandable body has a branched configuration and a third end, the method further comprising mounting the stent on a second guidewire so that the first guidewire extends between the first and second ends, and the second guidewire extends between the first and third ends.

22. A method as in claim 21, further comprising advancing the stent simultaneously over the first and second guidewires to the target location.

23. A method as in claim 21, wherein the target location includes a branched portion of the body lumen and wherein the first guidewire and the second guidewire are positioned in different branches of the branched portion of the body lumen, the method further comprising advancing the stent so that the second and third ends are disposed within the different branches of the branched portion of the body lumen.

24. A method as in claim 16, wherein the stent further comprises at least one constraining element configured to apply constraining force to assist in holding the at least one loop in alignment with the longitudinal axis, and wherein the method further comprises releasing the constraining force by affecting the at least one constraining element by a releasing mechanism.

25. A method as in claim 24, wherein the releasing mechanism comprises a mechanical force, electrical energy, a chemical reaction, an electrochemical reaction, thermal energy, radiofrequency, ultrasonic energy, infrared radiation, change in pH, or any combination of these.
